(19) United States
(12) Patent Application Publication
(10) Pub. No.: US 2025/0195859 A1
(43) Pub. Date: Jun. 19, 2025

(54) FLEXIBLE RESEALABLE SEPTUM

(71) Applicant: W. L. Gore & Associates, Inc., Newark, DE (US)

(72) Inventor: Jeffrey Towler, Newark, DE (US)

(21) Appl. No.: 18/984,807

(22) Filed: Dec. 17, 2024

Related U.S. Application Data
(60) Provisional application No. 63/734,674, filed on Dec. 16, 2024.
(60) Provisional application No. 63/611,357, filed on Dec. 18, 2023.

(51) Int. Cl. A61M 39/02

(52) U.S. Cl. CPC A61M 39/0208

(57) ABSTRACT

A medical device includes a tube and an occluding member. The tube is formed from an elastomeric material sufficiently rigid to maintain at least a portion of a lumen and sufficiently flexible to define an adjustable curvature along a tube axis. The occluding member is fixedly disposed within the tube lumen and is formed from an elastomeric material maintained in a compressed state by the tube. The occluding member includes an internal channel, the internal channel having a closed state maintained by the compressive force, preventing the flow of the fluid through the internal channel, and an open state wherein a pressure of the fluid overcomes the compressive force to at least partially open the internal channel to permit the flow of the fluid through the internal channel.

### CROSS REFERENCE TO RELATED APPLICATIONS

The present application claims priority to, and the benefit of, U.S. Provisional Patent Application No. 63/611,357, filed Dec. 18, 2023, and U.S. Provisional Patent Application No. 63/734,674, filed Dec. 16, 2024, the disclosure of each of which is incorporated herein in their entireties.

### FIELD

The present disclosure relates generally to implantable devices. More specifically, the disclosure relates to implantable subcutaneous ports and components incorporated therein.

### BACKGROUND

A subcutaneous port, also known as implantable venous port or port-a-cath, is a type of catheter port that is surgically implanted under the skin of a patient. The port is used for administering intravenous fluids and medicaments as well as to obtain fluid samples such as blood samples from the patient's body. The main components of the subcutaneous port are: a solid or rigid casing defining an internal chamber, a silicone septum defining a partition separating the internal chamber from an external environment, and a catheter extending from the casing. FIG. 1 shows an example of a port-a-cath 50 as known in the art, or more specifically, one end of such port-a-cath 50 that includes the access point 52. The other end, which is not shown, would include the open end of the catheter that is used to either administer fluids and medicaments to or obtain fluid samples from a target region in the patient's body. As shown in the figure, a catheter (“catheter”) 54 is attached to a solid or rigid casing (“casing”) 56 that is made of a rigid metal or hard plastic. The rigidity of the casing 56 provides a compressive force to an access point 52 which may be a septum made of silicone (“silicone septum”) when a needle is used to puncture the septum to reach a chamber (“internal chamber”) 58 that is inside the casing 56. During the puncturing process, the access point 52 is compressed by the rigid casing 56 so as to reduce the amount of fluid which may leak out from within the internal chamber 58.

However, solid or rigid casings 56 can have numerous problems or issues including but not limited to delayed complications such as infection occurring at the port-a-cath 50, device mispositioning, chronic inflammation, subcutaneous extravasation (leakage of chemotherapy agents from reservoir into surrounding tissue), etc. As such, there is a need for implantable subcutaneous ports that are designed to reduce such issues.

### SUMMARY

Disclosed herein are medical devices for controlling a flow of fluid passing through the device as well as methods of controlling fluid flow through such medical devices. Advantages of such devices and methods include flexible controlling of fluid flow through the devices as well as preventing or reducing a risk of fluid leaking through the medical device at unintended location or timing. In some examples, the advantages include providing a softer and more flexible implantable device, and furthermore, the implantable device includes a resealable septum that reduces leaking of fluid therethrough while maintaining flexibility of the device. Also, in various examples, the devices lack the metal or rigid plastic casing to provide additional comfort for the user when implanted.

According to one example (“Example 1”), a medical device for controlling a flow of a fluid passing through the device includes a tube and an occluding member. The tube has a tube length between opposing first and second tube ends and defining a tube axis of the tube. The first tube end has a first port and the second tube end having a second port. The first and second ports each communicates with a tube lumen extending within the tube between the first and second ports. The tube lumen defines a lumen diameter of the tube, the tube being formed from a first elastomeric material sufficiently rigid to maintain at least a portion of the lumen diameter and sufficiently flexible to define an adjustable curvature of the tube lumen disposed along the tube axis. The occluding member is fixedly disposed within the tube lumen between the first and second ports. The occluding member is formed from a second elastomeric material maintained in a compressed state by a compressive force from the tube. The second elastomeric material applies an expansive force against the tube within the tube lumen along at least a portion of the tube length. The occluding member further includes an internal channel extending through the occluding member between the first and second ports. The internal channel has a closed state maintained by the compressive force to collapse the internal channel to prevent the flow of the fluid through the internal channel and an open state overcoming the compressive force to at least partially open the internal channel to permit the flow of the fluid through the internal channel. In the closed state, the fluid disposed at the first port is at a first pressure that does not overcome the compressive force applied to the internal channel. In the open state, the fluid disposed at the first port is at a greater second pressure that overcomes the compressive force applied to the internal channel.

According to another example (“Example 2”) further to Example 1, the first and second elastomeric materials are different forms of a same material.

According to another example (“Example 3”) further to Example 1, the first and second elastomeric materials differ due to differing curing processes.

According to another example (“Example 4”) further to Example 1, the first elastomeric material defines an internal surface adjacent to the second elastomeric material.

According to another example (“Example 5”) further to Example 1, when the tube is cut to form a cut surface, the second elastomeric material at the cut surface protrudes axially outwardly at a greater degree than the first elastomeric material at the cut surface.

According to one example (“Example 6”), a flow-controlling medical device for controlling a flow of a fluid through the device, the device includes a containment member and an occluding member. The containment member defines a length extending between opposing ends of the containment member. The containment member further defines an inner lumen of the containment member extending between the opposing ends. The occluding member is disposed to occlude the inner lumen of the containment member. The occluding member is resealable such that a channel formed by a puncturing force applied to the occluding member will reseal to inhibit a passage of the fluid through the channel once the puncturing force is removed. The containment member is deformable to form a bent configuration of the device that maintains a resealability of the occluding member.

According to another example (“Example 7”) further to Example 6, the containment member defines a containment member axis. In the bent configuration, the containment member is displaced at least 30 degrees from the containment member axis.

According to another example (“Example 8”) further to Example 6, the containment member defines a containment member axis. In the bent configuration, and the containment member is displaced at least 30 degrees from an orthogonal axis that is orthogonal to the containment member axis.

According to another example (“Example 9”) further to Example 6, the containment member is deformable in that the medical device is reversibly deformable and is capable of returning to an initial configuration of the medical device.

According to another example (“Example 10”) further to Example 6, the bent configuration is a deformed configuration that maintains flow-controlling properties of the medical device.

According to another example (“Example 11”) further to Example 6, the bent configuration is nondestructive to an initial configuration of the medical device prior to the containment member being deformed to form the bent configuration of the device.

According to another example (“Example 12”) further to Example 6, the bent configuration does not form a folding of the medical device.

According to another example (“Example 13”) further to Example 6, the bent configuration is defined by a matching to an anatomy of a patient.

According to another example (“Example 14”) further to Example 6, the bent configuration is a loop configuration of the medical device.

According to another example (“Example 15”) further to Example 6, the containment member is a tube.

According to another example (“Example 16”) further to Example 14, the tube has the length of from 5 mm to 50 mm and a diameter of from 2 mm to 20 mm.

According to another example (“Example 17”) further to Example 6, the occluding member is configured to be radially compressed by the containment member.

According to another example (“Example 18”) further to Example 6, the occluding member is configured to be pressurized by the containment member.

According to another example (“Example 19”) further to Example 6, the containment member is an elastomeric material.

According to another example (“Example 20”) further to Example 6, the occluding member is an elastomeric material.

According to another example (“Example 21”) further to Example 19 or 20, the containment member and the occluding member are made of the same material undergoing different curing processes.

According to another example (“Example 22”) further to Example 21, the occluding member is made of uncured silicone, and the bendable containment member is made of cured silicone.

According to another example (“Example 23”) further to Example 6, the containment member has a greater stiffness than the occluding member.

According to another example (“Example 24”) further to Example 6, the medical device has a water leak pressure of from 10 mmHg to 20 mmHg.

According to another example (“Example 25”) further to Example 6, the containment member and the occluding member are dyed using different colors to visually distinguish between the containment member and the occluding member.

According to another example (“Example 26”) further to Example 6, an end portion of the medical device includes a guide member to visually distinguish between the occluding member and the containment member.

According to another example (“Example 27”) further to Example 6, the containment member and the occluding member are translucent or transparent.

According to one example (“Example 28”), a flow-controlling medical device for controlling a flow of a fluid through the device includes a containment member and an occluding member. The containment member defines a length extending between opposing ends of the containment member. The containment member further defines an inner lumen of the containment member extending between the opposing ends. The occluding member is disposed to occlude the inner lumen of the containment member.

The occluding member is resealable such that a channel formed by a puncturing force applied to the occluding member will reseal to inhibit a passage of the fluid through the channel when the puncturing force is removed. The occluding member includes a preformed internal channel extending through the occluding member and having a closed state where the internal channel is collapsed to prevent the flow of the fluid through the internal channel and an open state where the internal channel is opened to permit the flow of the fluid through the internal channel. The closed state is maintained when the fluid disposed at one of the opposing ends of the containment member is at a first pressure insufficient to open the internal channel. The open state is maintained when the fluid disposed at the one of the opposing ends of the containment member is at a second pressure sufficient to open the internal channel.

According to another example (“Example 29”) further to Example 28, the second pressure is a calibrated pressure that opens the internal channel upon reaching a threshold pressure.

According to another example (“Example 30”) further to Example 29, the threshold pressure is at least 40 psi.

According to another example (“Example 31”) further to Example 28, the occluding member is configured to be radially compressed by the containment member.

According to another example (“Example 32”) further to Example 28, the occluding member is configured to be pressurized by the containment member.

According to another example (“Example 33”) further to Example 28, the containment member is an elastomeric material.

According to another example (“Example 34”) further to Example 28, the occluding member is an elastomeric material.

According to another example (“Example 35”) further to Example 28, the containment member is a tube.

According to another example (“Example 36”) further to Example 35, the tube has the length of from 5 mm to 50 mm and a diameter of from 2 mm to 20 mm.

According to another example (“Example 37”) further to Example 28, the containment member is deformable to form a bent configuration of the device that maintains a resealability of the occluding member.

According to another example (“Example 38”) further to Example 37, the containment member defines a containment member axis. In the bent configuration, the containment member is displaced at least 30 degrees from the containment member axis.

According to another example (“Example 39”) further to Example 37, the containment member defines a containment member axis. In the bent configuration, the containment member is displaced at least 30 degrees from an orthogonal axis that is orthogonal to the containment member axis.

According to another example (“Example 40”) further to Example 37, the containment member is deformable in that the medical device is reversibly deformable and is capable of returning to an initial configuration of the medical device.

According to another example (“Example 41”) further to Example 37, the bent configuration is a deformed configuration that maintains flow-controlling properties of the medical device.

According to another example (“Example 42”) further to Example 37, the bent configuration is nondestructive to an initial configuration of the medical device prior to the containment member being deformed to form the bent configuration of the device.

According to another example (“Example 43”) further to Example 37, the bent configuration does not form a folding of the medical device.

According to another example (“Example 44”) further to Example 37, the bent configuration is defined by a matching to an anatomy of a patient.

According to another example (“Example 45”) further to Example 37, the bent configuration is a loop configuration of the medical device.

According to another example (“Example 46”) further to Example 28, the containment member has a greater stiffness than the occluding member.

According to another example (“Example 47”) further to Example 28, the containment member and the occluding member are made of the same material undergoing different curing processes.

According to another example (“Example 48”) further to Example 28, the medical device has a water leak pressure of from 10 mmHg to 20 mmHg.

According to another example (“Example 49”) further to Example 28, the containment member and the occluding member are dyed using different colors to visually distinguish between the containment member and the occluding member.

According to another example (“Example 50”) further to Example 28, an end portion of the medical device includes a guide member to visually distinguish between the containment member and the occluding member.

According to another example (“Example 51”) further to Example 28, the containment member and the occluding member are translucent or transparent.

According to one example (“Example 52”), a method of controlling a flow of a fluid through an elastomeric occluding member bounded by a containment member disposed to maintain a pressurized state of the occluding member, includes: applying a fluid pressure exceeding a rated pressure of the occluding member sufficient to open a preformed internal channel extending through the occluding member; directing the flow of the fluid through the opened preformed internal channel to an opposing end of the occluding member; and reducing the fluid pressure to close the preformed internal channel.

According to another example (“Example 53”) further to Example 52, the rated pressure is at least 4 psi.

According to another example (“Example 54”) further to Example 52, the rated pressure is at least 30 psi.

According to another example (“Example 55”) further to Example 52, the rated pressure is at least 70 psi.

According to another example (“Example 56”) further to Example 52, the rated pressure is at least 160 psi.

According to another example (“Example 57”) further to Example 52, the rated pressure is at least 190 psi.

According to another example (“Example 58”) a medical device for controlling a flow of a fluid passing through the device, the medical device comprising a first elastormeric layer; a second elastomeric layer adhered to the sealing layer on a second side of the sealing layer; and a channel a channel defined by the first and second elastomeric layers and the sealing layer; wherein the first and second elastomeric layers are configured to be in tension such that the first and second elastomeric layers compress the sealing layer; wherein the compression of the sealing layer prevents flow of fluid at a first pressure through a region of the channel that extends through the sealing layer; and wherein the compression of the sealing layer is insufficient to prevent flow of fluid at a second, higher pressure through the region of the channel that extends through the sealing layer.

According to another example (“Example 59”) further to Example 58, wherein the first and second elastomeric layers comprise a same material.

According to another example (“Example 60”) further to Example 58, wherein the first and second elastomeric layers comprise a same material.

According to another example (“Example 61”) further to Example 58, wherein a thickness of the sealing layer is less than about 50% of a thickness of the first elastomeric layer.

According to another example (“Example 62”) further to Example 61, wherein a thickness of the sealing layer is less than about 40% of a thickness of the first elastomeric layer.

According to another example (“Example 63”) further to Example 62, wherein a thickness of the sealing layer is less than about 20% of a thickness of the first elastomeric layer.

According to another example (“Example 64”) a flow-controlling medical device for controlling a flow of a fluid through the device, the device comprising: a rigid casing; a septum positioned in the casing; and an internal chamber defined by the casing, the septum, and the catheter; wherein the septum comprises: a first elastomeric layer; a sealing layer adhered to the first elastomeric layer on the first side of the sealing layer; a second elastomeric layer adhered to the sealing layer on a second side of the sealing layer; and a channel defined by the first and second elastomeric layers and the sealing layer; wherein the first and second elastomeric layers are configured to be in tension such that the first and second elastomeric layers compress the sealing layer, collapsing a region of the channel that extends through the sealing layer.

According to another example (“Example 65”) further to Example 64, wherein the compression of the sealing layer prevents flow of fluid at a first pressure through a region of the channel that extends through the sealing layer.

According to another example (“Example 66”) further to Example 65, wherein the compression of the sealing layer is insufficient to prevent flow of fluid at a second, higher pressure through the region of the channel that extends through the sealing layer.

According to another example (“Example 67”) further to Example 64 wherein the first and second elastomeric layers comprise a same material.

According to another example (“Example 68”) further to Example 64, wherein the sealing layer comprises the same material as the first and second elastomeric layers.

According to another example (“Example 69”) further to Example 64, wherein a thickness of the sealing layer is less than about 50% of a thickness of the first elastomeric layer.

According to another example (“Example 70”) further to Example 69, wherein a thickness of the sealing layer is less than about 40% of a thickness of the first elastomeric layer.

According to another example (“Example 71”) further to Example 70, wherein a thickness of the sealing layer is less than about 20% of a thickness of the first elastomeric layer.

According to another example (“Example 72”) a method of manufacturing a flow-controlling device, the method comprising: inserting a tool into a tube; compressing a first end of the tube around the tool; injecting an occluding material from the tool into the tube; compressing a second end of the tube to itself; injecting the occluding material into the tube; removing the tool from the tube; and curing the occluding material to form an occluding member.

According to another example (“Example 73”) further to Example 72, wherein compressing the first end of the tube seals the tube to the tool.

According to another example (“Example 74”) further to Example 72, wherein compressing the second end of the tube seals the tube.

According to another example (“Example 75”) further to Example 72, wherein compressing the second end of the tube to itself is performed after enough occluding material is injected into the tube that some occluding material leaks out of the second end.

According to another example (“Example 76”) further to Example 72, further comprising trimming the first end and the second end.

According to another example (“Example 77”) further to Example 76, further comprising performing a water leak test on the flow controlling device.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the embodiments of the disclosure and are incorporated in and constitute a part of this specification, illustrate examples, and together with the description serve to explain the principles of the disclosure.

FIG. 1 (prior art) is a line drawing of a port-a-cath device as known in the art;

FIG. 2A is a schematic diagram of a medical device according to embodiments disclosed herein;

FIG. 2B is a schematic diagram of the medical device of FIG. 2A further showing a needle puncturing through the medical device according to embodiments disclosed herein;

FIG. 2C is a schematic diagram of the medical device of FIG. 2A further showing a possible fluid flow pathway through the medical device according to embodiments disclosed herein;

FIG. 2D is a schematic diagram of the medical device of FIG. 2A further showing fluid flowing through the fluid flow pathway through the medical device according to embodiments disclosed herein;

FIG. 3A is a photograph of a side view of the medical device of FIG. 2A after being punctured to form a channel therein, according to embodiments disclosed herein (the image is to the scale shown in the image);

FIG. 3B is a photograph of a side view of an alternative medical device after being punctured to form a channel therein, according to embodiments disclosed herein (the image is to the scale shown in the image);

FIG. 4A is a photograph of a side view of alternative medical devices (the image is to the scale shown in the image);

FIG. 4B is a photograph of a top view of the alternative medical devices of FIG. 4A (the image is to the scale shown in the image);

FIG. 4C is a photograph of the top view of the alternative medical devices of FIG. 4A using alternative lighting as compared to FIG. 4B (the image is to the scale shown in the image);

FIGS. 5A and 5B are cross-sectional side views of alternative medical devices according to embodiments disclosed herein;

FIGS. 5C and 5D are front views of alternative medical devices according to embodiments disclosed herein;

FIG. 6 is a schematic diagram of a top view of a glaucoma treatment device according to embodiments disclosed herein;

FIG. 7 is a schematic diagram of a top view of an alternative glaucoma treatment device according to embodiments disclosed herein;

FIGS. 8A through 8H are schematic diagrams showing a process of forming the medical device according to embodiments disclosed herein;

FIG. 9 is a schematic diagram showing an assembly setup for performing a leak test on the medical device according to embodiments disclosed herein;

FIG. 10A (prior art) is an illustration of a top view of a prior-art glaucoma drainage device;

FIG. 10B (prior art) is an illustration of a side view of the prior-art glaucoma drainage device of FIG. 10A having a preformed curvature C-C;

FIG. 10C (prior art) is an illustration of an angled view of the prior-art glaucoma drainage device of FIG. 10A;

FIG. 11A is an illustration of a top view of a glaucoma drainage device according to embodiments disclosed herein;

FIG. 11B is an illustration of a side view of the glaucoma drainage device of FIG. 11A having a first curvature D-D according to embodiments disclosed herein;

FIG. 11C is an illustration of a side view of the glaucoma drainage device of FIG. 11A having a second curvature E-E according to embodiments disclosed herein;

FIG. 12 is a schematic diagram of an alternative medical device according to embodiments disclosed herein;

FIG. 13A is a schematic diagram of an operation of manufacturing the alternative medical device of FIG. 12 according to embodiments disclosed herein;

FIG. 13B is a schematic diagram indicating the internal stresses in the alternative medical device of FIG. 12 according to embodiments disclosed herein;

FIG. 14 is a photograph of a perspective view of the alternative medical device of FIG. 12 after having been punctured twice; and

FIG. 15 is a schematic cross-sectional slice of an alternative port-a-cath device according to embodiments disclosed herein.

It should be understood that some of the drawings and replicas of the photographs may not necessarily be shown to scale, unless otherwise indicated. In certain instances, details that are not necessary for an understanding of the disclosure or that render other details difficult to perceive may have been omitted. It should be understood, of course, that the disclosure is not necessarily limited to the particular examples or embodiments illustrated or depicted herein.

### DETAILED DESCRIPTION

### Definitions and Terminology

This disclosure is not meant to be read in a restrictive manner. For example, the terminology used in the application should be read broadly in the context of the meaning those in the field would attribute such terminology. Persons skilled in the art will readily appreciate that the various embodiments of the inventive concepts provided in the present disclosure can be realized by any number of methods and apparatuses configured to perform the intended functions. It should also be noted that the accompanying figures referred to herein are not necessarily drawn to scale, but may be exaggerated to illustrate various aspects of the present disclosure, and in that regard, the figures should not be construed as limiting. Some figures do, however, represent anatomy and the positioning of embodiments relative to that anatomy and such representations should be understood to be scaled and positioned accurately, with some deviation permitted as the anatomical structures depicted will vary in size and position from person to person.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

### Description of Various Embodiments

The present disclosure relates to systems, devices, and methods for providing a flow-controlling medical device that is reversibly deformable, or flexible, and also includes a resealable access point. In various examples, the access point is both resealable and permits fluid passage under certain fluid pressures. The flow-controlling medical device may achieve such operability without requiring a metal or solid casing to provide a compressive force.

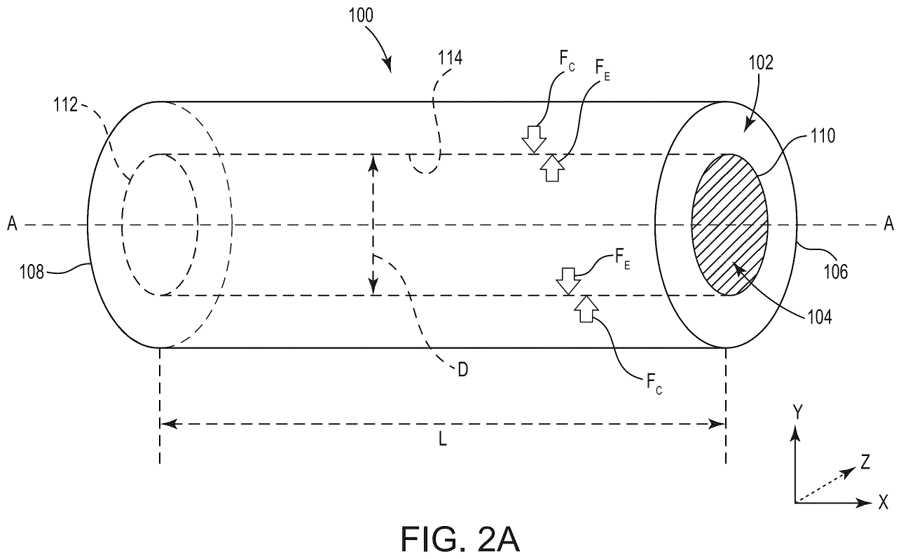

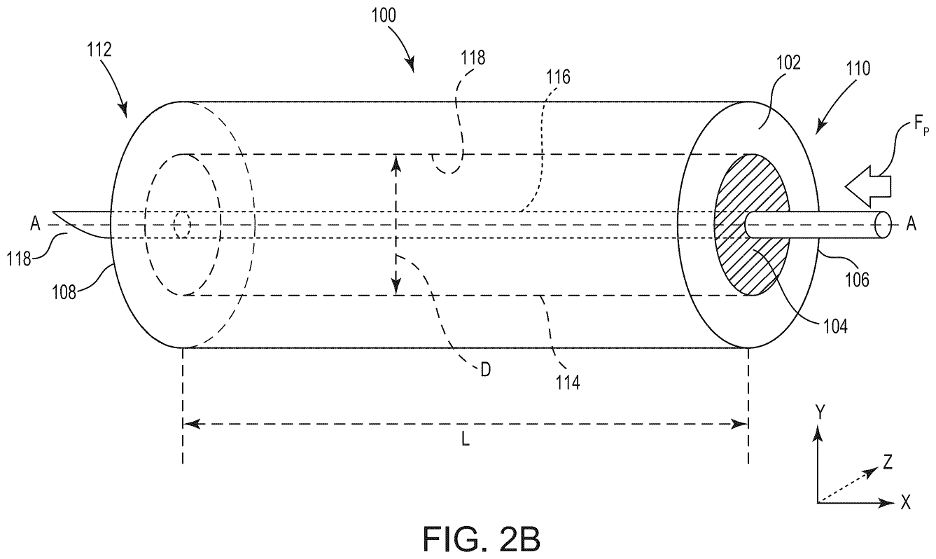

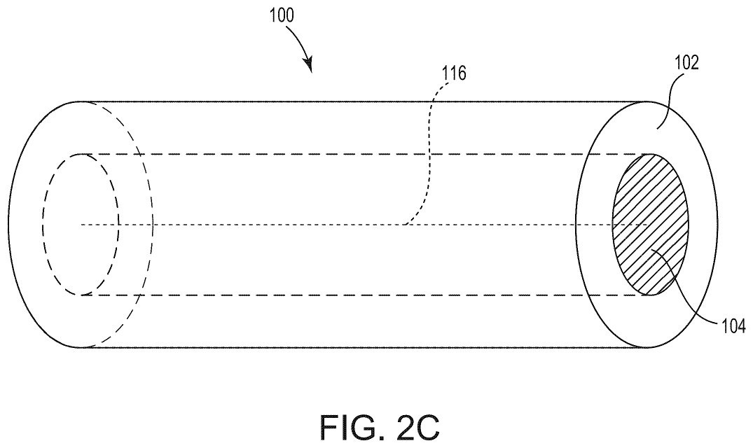

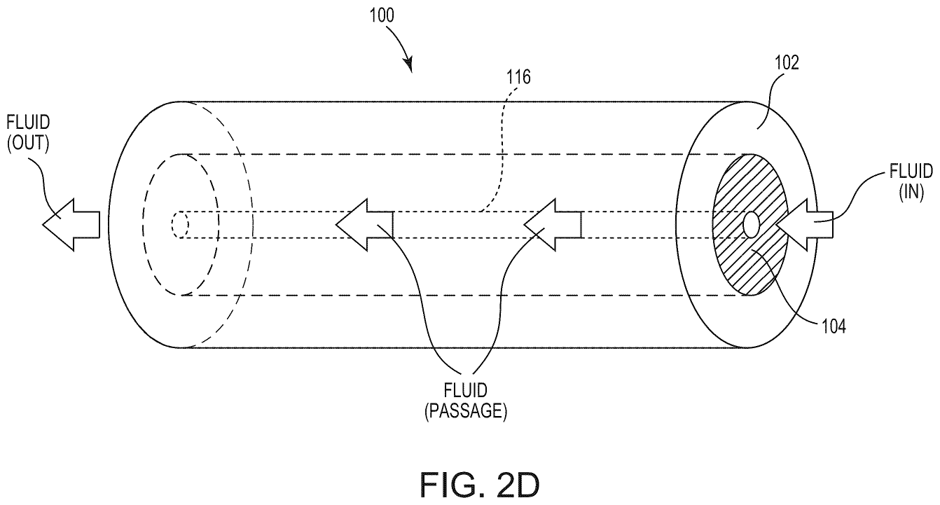

FIG. 2A illustrates an example of a medical device 100 for controlling a flow of a fluid passing through the device. In some embodiments, the medical device 100 can be referred to as a septum, an implantable device, a flow-controlling medical device (e.g., a flow stopper or a pressure regulator, depending on the configuration and pressures involved), or a port assembly, for example. The medical device 100 includes a tube 102 and an occluding member 104. The tube 102 is a containment member, and the occluding member 104 is a filler member or filler material, for example. The tube 102 defines a tube axis A-A, and the tube 102 has a tube length L defined between a first tube end 106 and a second tube end 108. In some examples, the tube length L is from about 5 mm to about 50 mm, and a diameter of the tube 102 is from about 2 mm to about 20 mm. In some examples, the tube 102 is stiffer than the occluding member 104.

The first tube end 106 has a first port 110, and the second tube end 108 has a second port 112. The first port 110 and the second port 112 may each communicate with a tube lumen 114 extending within the tube 102 between the first port 110 and the second port 112. The tube lumen 114 is an inner lumen that is defined by the tube 102 having a lumen diameter “D”. In the illustrated embodiment, the tube 102 is formed from a first elastomeric material that is sufficiently rigid to contain the occluding member 104 and sufficiently flexible to bend to accommodate various anatomical structures, for example, that would require curvature of the tube axis A-A.

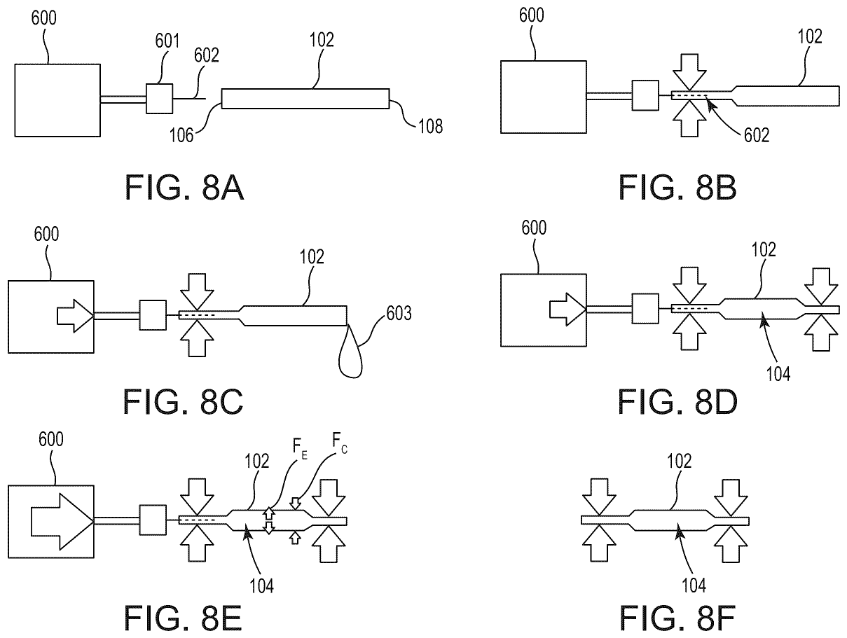

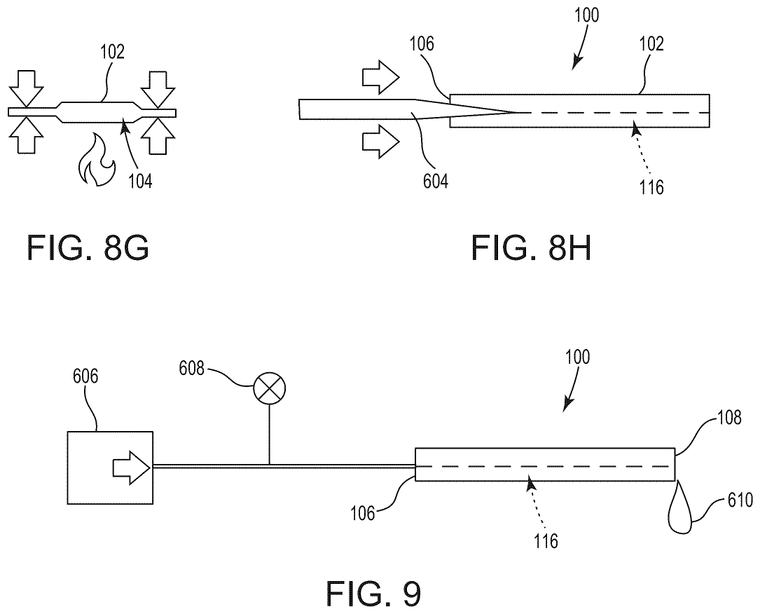

The occluding member 104 is fixedly disposed within the tube lumen 114 between the first port 110 and the second port 112, and the occluding member 104 is configured to occlude the tube lumen 114. The occluding member 104 is formed from a second elastomeric material that is maintained in a compressed state by the tube 102, as indicated by the compressive force F_{C}. As a reaction to being contained by the tube 102, the occluding member 104 applies an expansive force F_{E} against the tube 102 within the tube lumen 114 along at least a portion of the length L. The forces F_{C}, F_{E} are illustrated using thick arrows directed inwardly towards and outwardly from the occluding member 104, respectively. The forces F_{C}, F_{E} are established during the manufacturing of the medical device 100, which is described later with respect to FIGS. 8A-8H. In short, the medical device 100 is made by forcing the occluding member 104 into the tube 102. The force causes the tube 102 to expand and the occluding member 104 to compress, so the forces F_{C}, F_{E} remain in a static balance.

In some examples, the first and second elastomeric materials are two different materials. In some examples, the first and second elastomeric materials are different forms of a same material. When they are the same material, the first and second elastomeric materials may be formed using different manufacturing (e.g., curing) processes, causing the difference in physical properties therebetween. In some examples, the first elastomeric material of the tube 102 defines an internal surface adjacent to the second elastomeric material of the occluding member 104. In some examples, when the tube 102 is cut to form a cut surface (or otherwise formed), the second elastomeric material at the cut surface (or other end surface of the tube 102) protrudes axially outwardly at a greater degree than the first elastomeric material at the cut surface. In different terms, the occluding member 104 may be configured to project longitudinally beyond the tube 102. Other possible materials that may be used for the elastomeric material may include but are not limited to one or more of the following: silicone, neoprene, polybutadiene, polyisoprene, styrene-butadiene block copolymers, nitrile rubber, polyurethane, ethylene-propylene rubber, fluoroelastomers such as Viton™, or poly(styrene-block-isobutylene-block styrene), for example.

As used herein, the medical device 100 begins in a sealed state in which there is no channel 116 formed therein. However, as shown in FIG. 2B, during use of the medical device 100, a tool 118 (e.g., a 30-gauge needle) can be used to puncture through the medical device 100, for reasons that will be discussed below. When the puncturing force FP is applied to the tool 118 in the axial direction (e.g., generally along the A-A axis shown in FIG. 2A), for example by a physician or a user of the device 100 before the device 100 is applied to a patient during a procedure or surgery, the channel 116 is formed by the tool 118. The channel 116 extends from one end (one of 106 and 108) toward the other end (the other of 106 and 108). After removing the tool 118 from the channel 116, the compressive force F_{C} from the tube 102 causes the occluding member 104 to be in a resealed state in which the channel 116 is collapsed. Such a closed state is shown in FIG. 2C

In some embodiments, the medical device 100 is configured and/or positioned to block the flow of fluid from the first tube end 106 to the second tube end 108 and/or from the second tube end 108 to the first tube end 106. The collapsing of the channel 116 inhibits fluid from flowing through the medical device 100. However, if the fluid pressure at one of the tube ends 106, 108 rises beyond a threshold, the channel 116 will be opened to some extent, as shown in FIG. 2D. In such an opened state, the medical device 100 will allow the fluid to pass through from the higher-pressure tube end 106, 108 to the lower-pressure tube end 106, 108, respectively. The pressure threshold at which the medical device 100 opens can vary depending on, for example, the material(s) of the tube 102 and occluding member 104, the length L, the physical characteristics of the fluid, and the value of the forces F_{C}, F_{E}. With respect to the forces F_{C}, F_{E}, the pressure threshold at which the medical device 100 opens is related to the forces used during the manufacturing of the medical device 100.

In some embodiments, water leak pressure is an important characteristic of the medical device 100. Water leak pressure is defined as the amount of pressure necessary for water to pass (or leak) through the channel 116 from one tube end 106, 108 of the medical device 100 to the other tube end 106, 108. Once the pressure was dropped below the water leak pressure level, then the channel 116 will collapse and the medical device 100 will again be in a closed state.

Table 1 below provides the different water leak pressures measured for a medical device 100 with a length L of about 2 mm after being punctured using a 30-gauge tool 118 (such as a needle or wire) having a diameter of 0.3112 mm (0.01225 in.). The water leak pressures are correlated with different pressures maintained by a dispense assembly (e.g., the dispense assembly 600 as further discussed herein) that is used to form the medical device 100. The pressure of the dispense assembly dictates the force at which the occlusion member 104 is inserted into the tube 102. As shown, the greater the pressure that is maintained by the dispense assembly, the greater the water leak pressure becomes. As the pressure maintained by the dispenser assembly increases from virtually zero pressure to 40 psi, 50 psi, 70 psi, and 90 psi, the water leak pressure that is required for water to reopen the channel 116 and pass through the channel 116 also increases to be greater than 30 psi, greater than 70 psi, greater than 160 psi, and greater than 190 psi, respectively.

TABLE 1
Comparison of machine pressure setpoint of a dispenser assembly and
the corresponding water leak pressure required to pass water
from one tube end 106, 108 to the other.

| Machine pressure setpoint of | Water leak pressure |
|---|---|
| dispenser assembly (psi) | (psi) |
| 0 | 4 |
| 40 | 31 |
| 50 | 77 |
| 70 | 165 |
| 90 | 193 |

Table 1 may pertain to some of the embodiments disclosed herein, for example a vascular implant which may be implanted inside a patient's vasculature to operate as a valve which allows fluid (e.g., aqueous humor or blood) flow only if the fluid pressure exceeds a threshold pressure (which may be the same as, similar to, or different from the water leak pressure). In some examples, the medical device 100 has a water leak pressure of from 5 psi to 10 psi, for example, when the medical device 100 is implemented or implanted. In some examples, the water leak pressure may be from 10 psi to 20 psi, from 20 psi to 30 psi, from 30 psi to 40 psi, from 40 psi to 50 psi, from 50 psi to 100 psi, from 100 psi to 150 psi, from 150 psi to 200 psi, or any other suitable range therebetween.

In some examples, the tube 102 is elastically or plastically deformable to form a bent configuration wherein the medical device 100 retains its flow controlling capabilities. In some examples, the tube 102 is pre-bent or pre-formed with a bend or curvature such that the tube axis A-A at the tube end 108 is oriented at least 30 degrees away from the tube axis A-A at the tube end 106. In some examples, the tube axis A-A may be bent, curved, or otherwise displaced at least 35 degrees, 40 degrees, 45 degrees, 50 degrees, 55 degrees, 60 degrees, 65 degrees, 70 degrees, 75 degrees, 80 degrees, 85 degrees, 90 degrees, 100 degrees, 110 degrees, 120 degrees, 130 degrees, 140 degrees, 150 degrees, 160 degrees, 170 degrees, 180 degrees, 200 degrees, 220 degrees, 240 degrees, 260 degrees, 270 degrees, 300 degrees, 330 degrees, or any other suitable range or value therebetween. For example, the displacement may form a loop according to the range of degree as mentioned above.

In some examples, the tube 102 and/or occlusion member 104 are elastically deformable such that the medical device 100 deflects when acted on by an external force and will return to its initial configuration after the external force is removed. In some examples, the tube and/or occlusion member 104 are repeatedly plastically deformable in that the medical device 100 is configured to be shapable by, for example, a physician applying an external force to the medical device 100. In such examples, after the external force is removed, the medical device 100 will mostly maintain its new configuration. The medical device 100 is also configured to be returnable to its initial configuration and/or changeable to another configuration, for example, by the physician applying different forces to the medical device 100. In some examples, the medical device 100 includes a plastically deformable support (e.g., a spine extending longitudinally through the tube 102 and/or the occlusion member 104) that facilitates the shapability of the medical device 100. In some examples, when the medical device 100 is in a temporary or semi-permanently bent or curved configuration, the medical device 100 maintains its flow-controlling properties.

In some examples, a bent or curved configuration does not cause folding or kinking of the medical device 100, nor does such a configuration cause the medical device 100 to form a permanent fold, kink, or pleat. In some examples, a bent or curved configuration that is defined by the device 100 partially or fully accommodates and/or matches an anatomy of a patient. In some examples, the medical device 100 bends to accommodate an inner vasculature or an organ of the patient which the medical device 100 is in contact with.

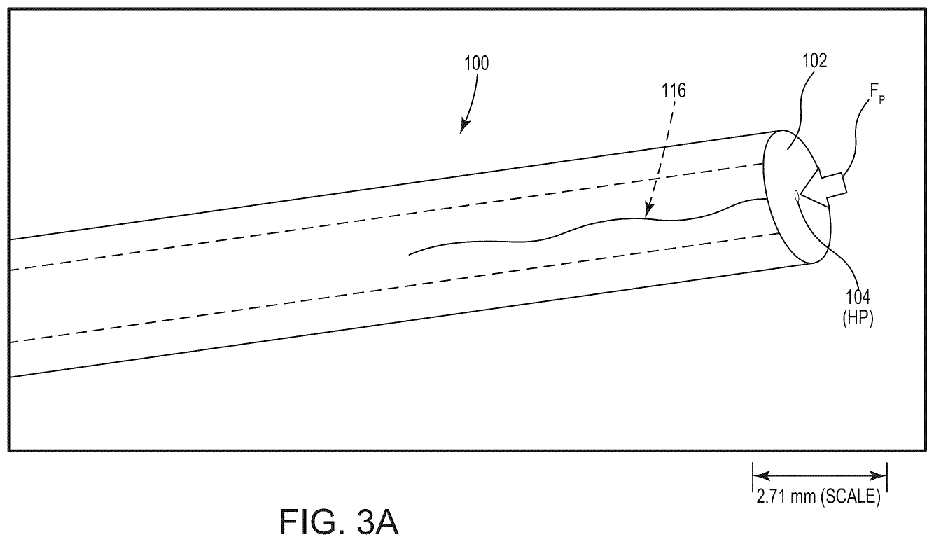

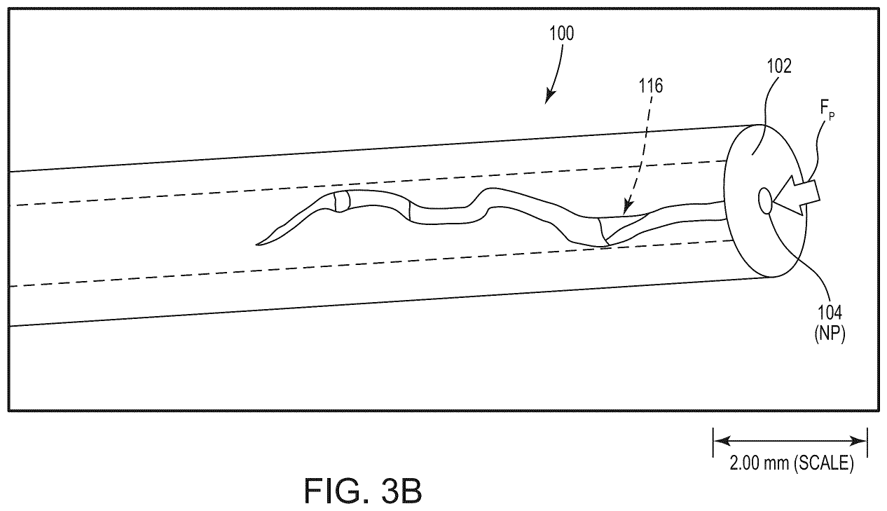

FIGS. 3A and 3B show the difference in the medical device 100 when the occluding member 104 is formed using different amounts of pressure, such that the occluding member 104 is being compressed different amounts by the tube 102. In both figures, the occluding member 104 is disposed with the tube 102 and has been punctured with a puncturing force FP using the tool 118 to form the internal channel 116. The tool 118 has been removed prior to taking the images. In FIG. 3A, the occluding member 104 was formed at high pressure (HP), so the occluding member 104 is in an HP state. Thus, the internal channel 116 is nearly invisible to the naked eye. However, in FIG. 3B, the occluding member 104 was under virtually no pressure (NP) when it was formed, so the occluding member 104 is in an NP state. Thus, the internal channel 116 is visible to the naked eye and allows water to pass through into the internal channel 116 at a relatively low pressure, resulting in a lower water leak pressure than in the example of FIG. 3A. The low water leak pressure of the example of FIG. 3B is caused by the tube 102 exerting an insubstantial amount (if any) of compressive force on the occluding member 104.

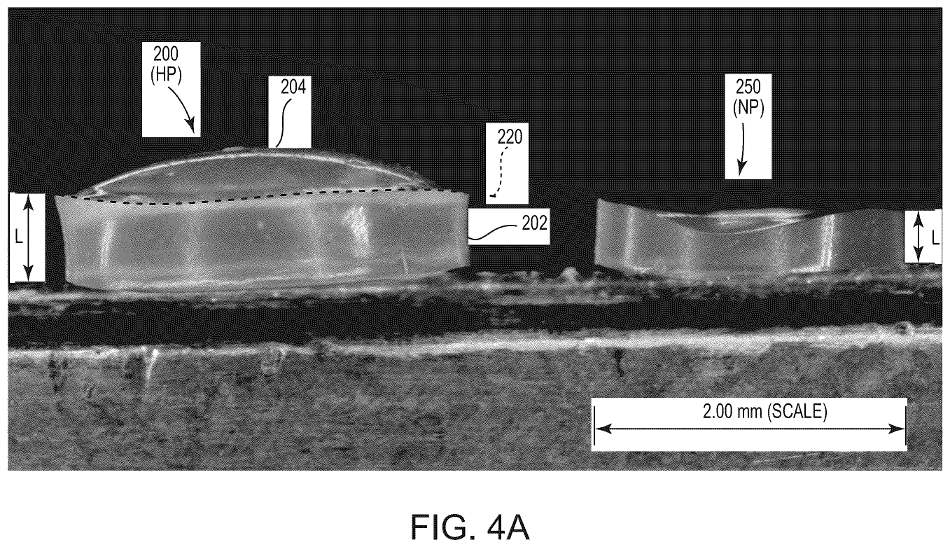

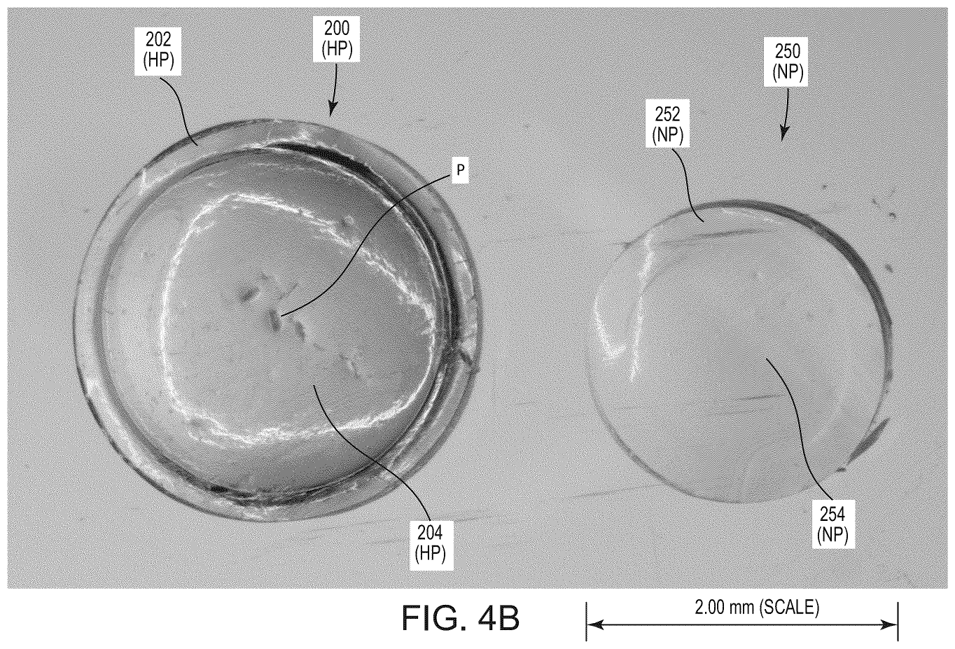

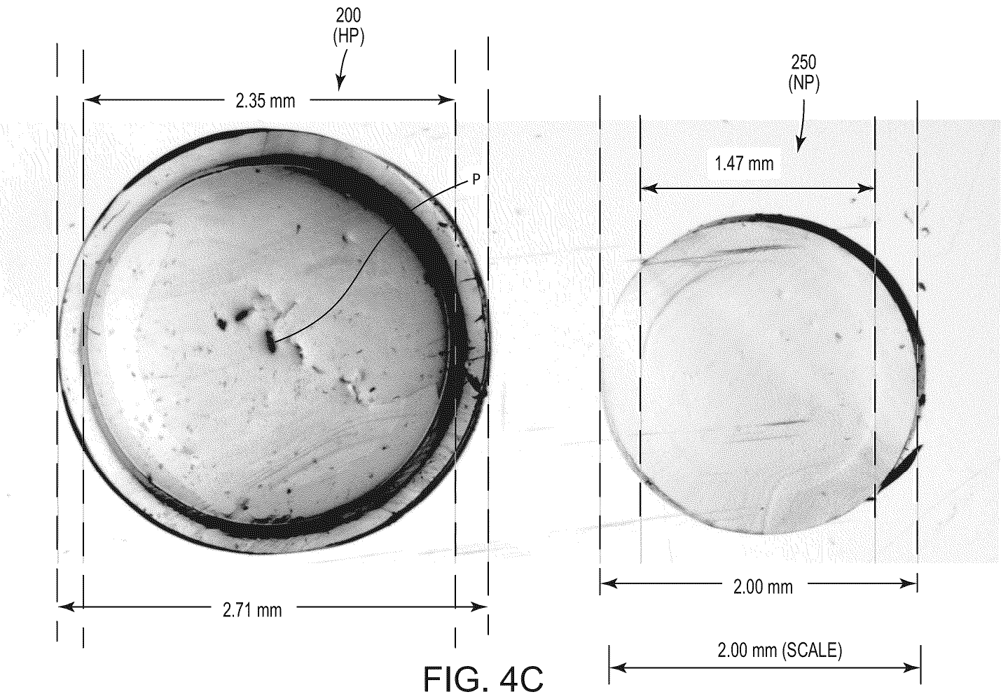

FIGS. 4A through 4C show alternative medical devices 200, 250 that each have significantly shorter longitudinal lengths L as compared to the device 100 presented in FIGS. 2A-2D. The medical device 200 in which the occluding member 104 is in an HP state is shown on the left, and the device 250 in which the occluding member 104 is in an NP state is shown on the right, in each of FIGS. 4A-4C. As shown in FIGS. 4B and 4C, the site of puncture P (also corresponding to the location of the channel 116) is visible in the HP example, whereas there is no puncture in the NP example. In some examples, due to the HP state that the medical device 200 was formed in, the tube 202 has an outer diameter of 2.71 mm, and the occluding member 204 has an outer diameter of 2.35 mm. In some examples, due to the NP state that the medical device 250 was formed in, the tube 252 has an outer diameter of 2.00 mm, and the occluding member 254 has an outer diameter of 1.47 mm.

In some examples, the medical devices 200, 250 are formed by initially creating a longer device (a la the medical device 100, shown in FIG. 2D) and then cutting a thin slice therefrom. Evidence of such a process includes, in some examples, a cut surface 220 (shown by the dotted line in FIG. 4A). Due to the HP state of the medical device 200, the occluding member 204 experiences the Poisson effect. Thus, as the tube 202 radially squeezes the occluding member 204, the occluding member 204 expands longitudinally outward, beyond the longitudinal ends of the tube 202. However, due to the NP state of the medical device 250, the occluding member 254 is the same length L as the tube 252 (since the tube 252 is exerting little to no radial pressure on the occluding member 254).

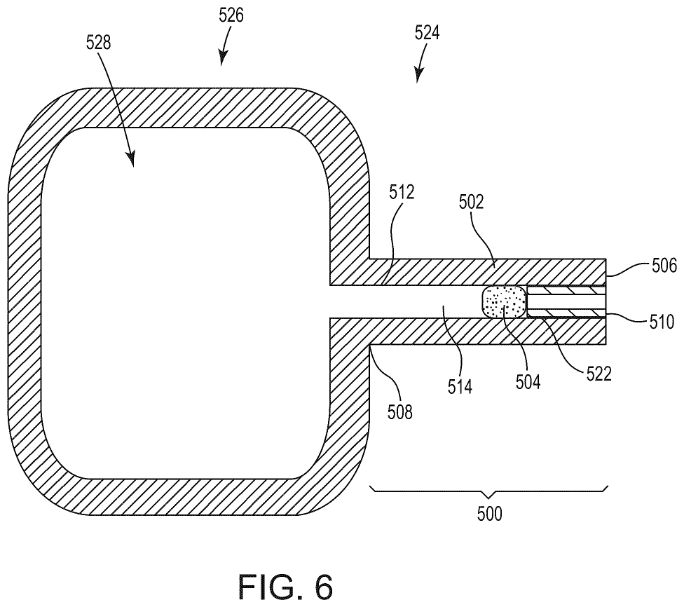

Due to the substantially shorter length L of the medical devices 200, 250, the water leak pressures will be on a very different scale than that of the medical device 100 (shown in FIG. 2D), as described in Table 1. In some examples, the medical devices 200, 250 are configured to have a water leak pressure from about 10 mmHg to about 20 mmHg, for example, when the medical devices 200, 250 is implemented in a glaucoma drainage implant (e.g., as shown in FIG. 6). In some such examples, the water leak pressure is from 10 mmHg to 13 mmHg, from 13 mmHg to 15 mmHg, from 15 mmHg to 18 mmHg, from 18 mmHg to 20 mmHg, or any other suitable range therebetween.

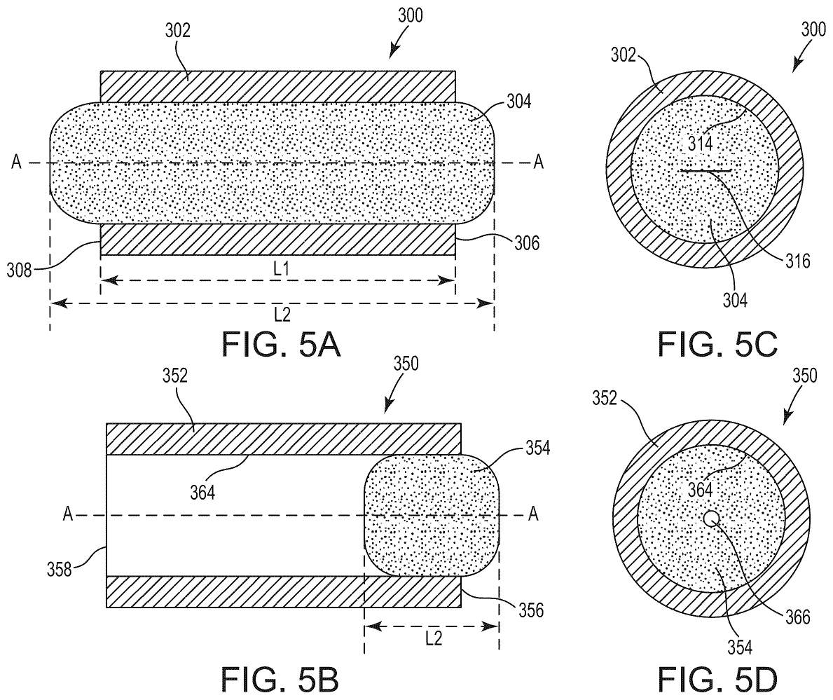

FIGS. 5A and 5B show two examples of the medical device 300, 350, respectively. The medical devices 300, 350 each include a tube 302, 352 and an occluding member 304, 354, respectively. In the illustrated embodiments, and one or more portions of the occluding members 304, 354 protrude or extend past one or more ends 306, 308, 356, 358 of the tubes 302, 352, respectively. Each of the tubes 302, 352 has a longitudinal length of L1 (measured along the longitudinal axis A-A as shown), and the occluding members 304, 354 has a longitudinal length of L2 which is different from the length L1, respectively.

In some examples, he portion(s) of the occluding members 304, 354 protruding or extending past one or more tube ends 306, 308, 356, 368 are caused by the Poisson Effect as a consequence of the pressure or force being applied to the occluding members 304, 354 by the tubes 302, 352. In some examples, the pressure or force being applied by the tubes 302, 352 is in reaction to the occluding members 304, 354 being inserted at high pressure, respectively. In some examples, the tubes 302, 352 are positioned around the occluding members 304, 354 and then the tubes 302, 352 are cured or otherwise treated (e.g., thermally, chemically, and/or mechanically) which causes the tubes 302, 354 to shrink and apply pressure to the occluding members 304, 354, respectively, thereby causing the aforementioned protrusion or extension.

In FIG. 5A, the length L2 is greater than the length L1, and the occluding member 304 protrudes or extends past both the tube ends 306 and 308 of the tube 302. In FIG. 5B, the length L2 is less than the length L1, and the occluding member 354 protrudes or extends past only one tube end 356, 358 (in the example shown, the tube end 356) of the tube 352. The occluding member 354 therefore occupies only a portion of the lumen 364 within the tube 352 such that a portion of the lumen 364 remains unoccupied by the occluding member 354.

FIGS. 5C and 5D show two examples of the medical device 300, 350, respectively. The occluding members 304, 354 occupy the lumens 314, 364 of the tubes 302, 352, respectively. As shown in FIG. 5C, the occluding member 304 includes a channel 316 with a slit shape, and as shown in FIG. 5D, the occluding member 354 includes a channel 366 with a circular or round hole. Other shapes of channels can be made in an occluding member, such as, for example, bent-shaped, curved, polygonal, etc. In some examples, the punctures that create the channels 316, 366 are pre-formed, and in some examples, the punctures that create the channels 316, 366 are formed in situ (i.e., after implantation of the medical devices 300, 350). In some examples, the channels 316, 366 extend the entire length L2 of the occluding members 304, 354, and in some examples, the channels 316, 366 are pre-formed to initially extend only part of the way through the length L2 of the occluding members 304, 354. In some examples, the channels 316, 366 are normally closed absent the presence of a tool (e.g., the tool 118, shown in FIG. 2B).

FIG. 6 shows a medical device 500 that has been integrated into a glaucoma drainage implant. In some examples, the medical device 500 includes a tube 502, an occluding member 504, a first tube end 506, a second tube end 508, a first port 510, a second port 512, and a lumen 514, which can be similar to or the same as the corresponding components in the previously described medical devices 100, 200, 300, and/or 350.

In some examples, the medical device 500 further includes a guide member 522 that is positioned inside of the lumen 514 between a proximal end of the occluding member 504 and the first port 510. In some examples, the guide member 522 is colored to visually distinguish the tube 502 from the occluding member 504. Thus, the guide member 522 helps the physician to avoid puncturing the medical device 100 at an incorrect location, such as causing a puncture at the tube 502 instead of the occluding member 504, for example, in the process of forming the channel 516, in the process of supplying/refilling a medical assembly 524, and/or releasing/extracting fluids/objects from the medical assembly 524.

In some examples, the tube 502 and the occluding member 504 are both transparent, both translucent, or one or the other may be transparent/translucent. In some examples, one of the tube 502 and the occluding member 504 is more transparent (i.e., have a greater level of transparency) than the other, in order to assist in visually distinguishing the two members. In some examples, to assist in making the tube 502 and the occluding member 504 to be more visually distinguishable, there may be dyes with different colors (not shown) being used to dye the different members. In some such examples, the elastomeric material of the tube 502 that defines the lumen 514 is a different color from the adjacent, elastomeric material of the occluding member 504 which makes the borders therebetween more distinct.

In some examples, the medical device 500 is part of the medical assembly 524 which includes a body portion 526 that defines therein a reservoir 528 which is fluidly coupled with the second port 512 of the lumen 514. In some examples, the tube 502 is a separate component that is attached to the body portion 526. In some examples, the body portion 526 and the tube 502 are formed of a single unitary piece of material or construct. The occluding member 504 is shown disposed within the lumen 514 and functions as a septum between the first port 510 and the reservoir 528. In some examples, the occluding member 504 is a plug to prevent fluid from passing between the ports 510 and 512.

In some examples, the occluding member 504 is a regulating or pressure release valve that controls the pressure of a fluid at the first port 510 and/or in the reservoir 528. As such, the occluding member 504 is opened or closed based upon the difference between the fluid pressures at the ports 510 and 512. In some examples, the medical device 500 is an implantable glaucoma drainage and/or drug delivery device. The glaucoma drainage devices and glaucoma drug delivery devices as referred to herein may include one or more of the devices described and shown in U.S. Patent Publication Nos. 2023/0142433 and 2023/0142430 and U.S. patent Ser. No. 63/700,363 (W.L. Gore & Associates, Inc.), the disclosures of which are incorporated herein by reference in their entireties for all purposes. In cases where the medical device 500 is used as a valve and is positioned in an inlet of a drainage device, the occluding member 504, when implanted at least partially inside the eye of a patient, opens when the anterior chamber's intraocular pressure (IOP) exceeds a predetermined threshold pressure of the valve. In the open configuration, fluid passage is permitted through the internal channel 516, as shown in FIG. 2D for example. In other cases where the medical device 500 is used as a stopper and is positioned in a refill tube of a drug delivery device, the occluding member 504, when implanted at least partially inside of the eye of a patient, remains closed until it is opened by a physician using a tool (not shown) to add drug treatment media to the reservoir 528.

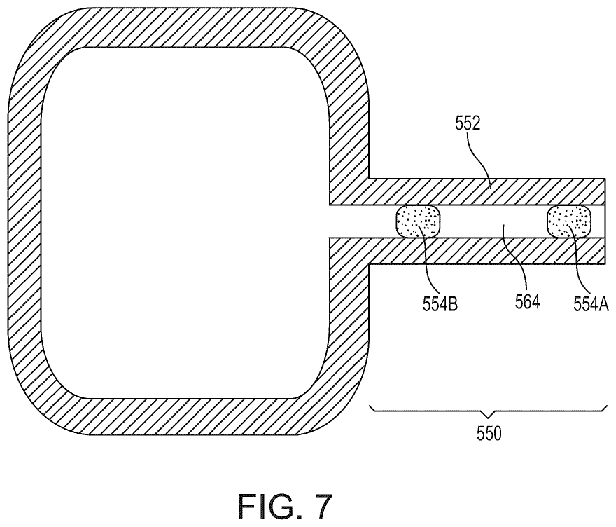

FIG. 7 shows the medical device 550 having a plurality of occluding members 554A and 554B in the lumen 564 of the tube 552. Although there are only two shown in the figure, in some examples, there may be three or more such occluding members 554 as suitable. The occluding member 554A is disposed closer to the tube end 556, and the occluding member 554B is disposed closer to the other tube end 558. In some examples, the occluding members 554A and 554B have different physical properties such as different water leak pressures that are required for a fluid to overcome the compressive force from the tube 552 and pass through the internal channel (not shown) formed therein. For example, the minimum pressure required for fluid to pass through the occluding member 554A can be greater than the minimum pressure required of the occluding member 554B, or vice versa.

FIGS. 8A-8H illustrate a process of making the device 100 according to examples disclosed herein. FIG. 8A shows an initial setup of a dispenser assembly 600 with a tool, such as a needle 602, being prepared to be inserted into the tube 102, which may be a silicone tubing. The dispenser assembly 600 is a device that is prefilled with a material that is to be used as the occluding member 104, for example silicone and/or any other suitable polymer (hereinafter referred to as an occluding material 603, shown in FIG. 8C). The needle 602 may be connected to a dispenser attachment 601 which is fluidly coupled with the dispenser assembly 600. In FIG. 8B, the needle 602 is inserted into the tube end 106 (or the tube end 108) of the tube 102, and a radial pressure and/or compression is applied to the tube end 106 of the tube 102 (shown by the vertical arrows), for example via the use of a clamp or any suitable affixing device, so as to seal and/or tighten the tube end 106 to prevent any of the inserted occluding material 603 from escaping (e.g., via backflow).

In FIG. 8C, while the tube end 106 remains sealed around the needle 602, the dispenser assembly 600 is activated to inject the occluding material 603 (shown by the horizontal arrow) into the tube 102. The injection continues until the occluding material 603 leaks through the opposing tube end 108 of the tube 102 as shown. In FIG. 8D, while the dispenser assembly 600 is activated, the tube end 108 of the tube 102 is also sealed, tightened, and/or compressed via a pressure applied thereto (shown by the second set of vertical arrows) such that the two ends 106 and 108 are both sealed or tightened to prevent the occluding material 603 from leaving the tube 102, effectively forming therein the occluding member 104.

In FIG. 8E, the dispenser assembly 600 increases the force (or pressure) with which it is injecting the occluding material 603 into the tube 102 (shown by the larger horizontal arrow) such that the continuous injection at a greater force (or pressure) causes the internal occluding material 603 to exert the expansive force F_{E} as shown by the smaller, outward-facing vertical arrows. The expansive force F_{E} is opposed by the compressive force F_{C} from the tube 102 as shown by the smaller, inward-facing vertical arrows. In some examples, the compressive force F_{C} is initially smaller than the expansive force F_{E}, so the tube 102 expands as it is filled with the occluding material 603. Once the filling has ceased, the tube 102 stops expanding and the compressive force F_{C} and the expansive force F_{E} reach equilibrium. In FIG. 8F, the tube 102 is removed from the dispenser assembly 600 while still maintain the pressure applied to the ends 106 and 108.

In FIG. 8G, the tube 102 and the occluding member 104 are placed in a heating assembly such as a convection oven. The tube 102 and the occluding member 104 are heat-treated at a predetermined temperature for a predetermined period of time. In some examples, the predetermined temperature may be about 120 degrees Celsius (° C.), and the predetermined period of time may be about 30 minutes. Thereafter, the tube 102 and the occluding member 104 are removed from the heating assembly and thereafter cooled, and the tube ends 106, 108 are trimmed to any suitable configuration or length. In FIG. 8H, a needle 604 is inserted through the occluding member 104 from the tube end 106 and subsequently removed, thereby forming the internal channel 116 of the medical device 100.

In FIG. 9, the medical device 100 that is formed according to the process shown in FIGS. 8A-8H undergoes a leak test using the assembly as shown. The assembly for the leak test includes a micropump 606 and a pressure sensor 608 that is coupled with the micropump 606. The micropump 606 is fluidly connected to the internal channel 116 of the medical device 100 at the tube end 106 and is activated to dispense water 610 into the channel 116. As the water 610 leaves the channel 116 through the other tube end 108 of the medical device 100, the pressure reading is recorded as shown by the pressure sensor 608. The measurement may be in the unit of psi, an example of which is shown in the right column of Table 1, or in the unit of mmHg, whichever is more suitable for the implementation of the medical device 100.

According to some examples, the tube 102 may be a silicone tubing which has an outer diameter of 0.025 inch (0.635 mm) and an inner diameter of 0.012 inch (0.305 mm) and is manufactured by SMI Manufacturing Inc. (Saginaw, MI). The occluding material forming the occluding member 104 may be a silicone adhesive such as a two-part silicone adhesive MED2-4213 manufactured by Avantor, Inc. (Allentown, PA). The dispenser assembly 600 may be any suitable fluid dispensing workstation such as EFD Ultra® 2400 Series Dispensing Workstation provided by Nordson Corporation (Westlake, OH). The dispenser attachment 601 may be an HPx High-Pressure Dispensing Tool using 5 cc Nordson EFD syringe barrels and pistons, all of which are provided by Nordson Corporation (Westlake, OH), for example. The needle 602 connected to the dispenser attachment 601 may be a 25-gauge general purpose needle such as Nordson EFD part no. 7018345 dispensing sip, provided by Nordson Corporation (Westlake, OH), for example.

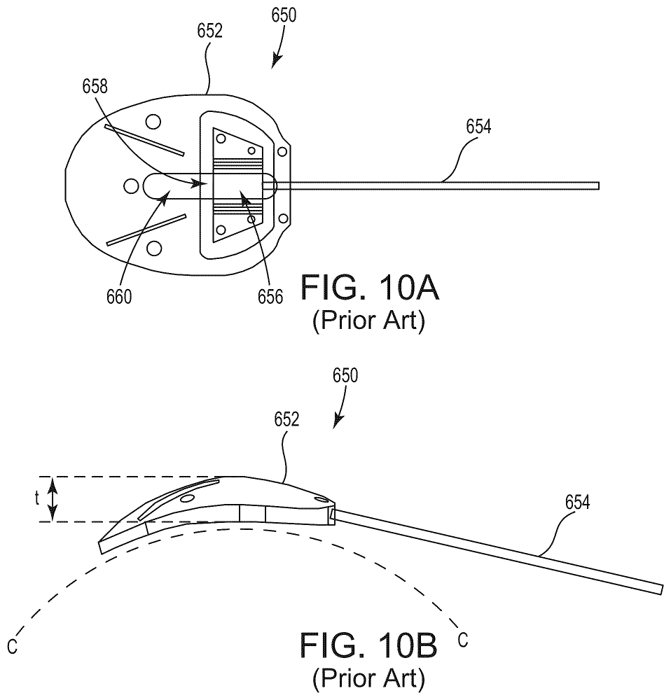

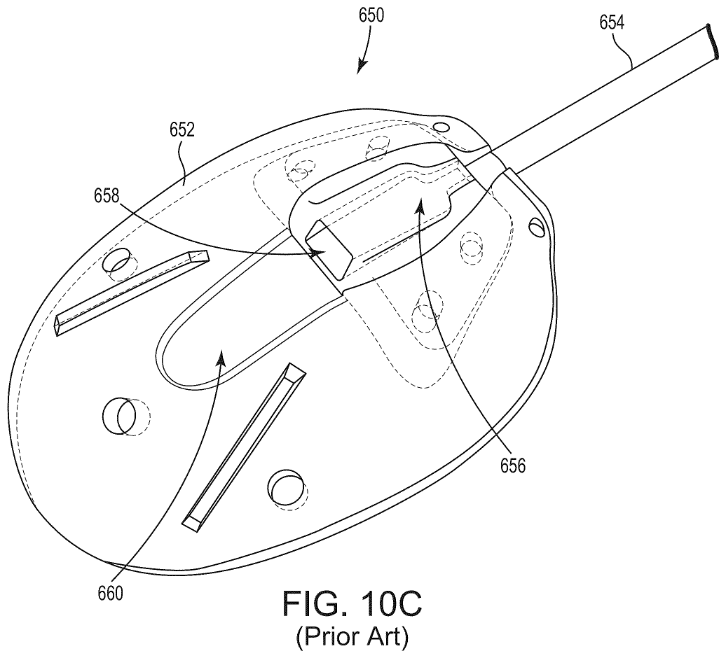

Some prior-art drainage devices may implement solid shells or plates, such as those made from silicone as shown in FIGS. 10A-10C, to receive fluid from the anterior chamber (AC) of the eye and store it inside an internal chamber formed between the plates.

FIGS. 10A-10C illustrate a glaucoma drainage device 650 as known in the art. The device includes a plate body 652 which defines a surface over which the drained fluid (aqueous humor) is directed to flow, and a drainage tube 654 which directs the fluid (aqueous humor) to flow over the surface of the plate body. The implanted plate body 652 of FIGS. 10A-10C defines a surface area for the fluid to be absorbed by the surrounding tissue, more specifically by the conjunctival tissue on the superior side of the plate body. In the art, this surface area is referred to as a “bleb”, and the absorption of redirected fluid into the surrounding tissue is also referred to as filtering through the tissue, hence these devices are sometimes referred to as “filtration” devices. The plate body 652 has a maximum thickness t which in the example shown is 2.1 mm, and is made of medical-grade silicone which lacks flexibility to conform to the curvature of the eye when implanted. As such, the plate body B has a preformed curvature (defined by the broken line C-C in FIG. 10B) to approximate the curvature of the surface of the eye. The curvature C-C is fixed and is the same for all glaucoma drainage devices, and thus, it is believed, fails to accommodate the unique curvature of each patient's eye. The plate body 652 also includes a valve 656 embedded therein, with an outlet 658 which directs the fluid from the tube 654 toward a gutter 660 located on an outer surface of the plate body 652 as illustrated in FIG. 10C.

In the prior-art device of FIGS. 10A-10C, which is an Ahmed® Glaucoma Valve model FP7 (New World Medical, Inc.; Rancho Cucamonga, CA), the plate body 652 has a structure that is stiff to allow the user to hold any part of the plate body 652 using tools such medical tweezers, hemostats, or any other suitable medical tool, so the user can push the plate body 652 into the tissue pocket in the eye which is made by the incision, while holding the plate body 652 with the medical tool. Likewise, the prior-art device of FIGS. 10A-10C relies upon a slippery or low-friction surface of the device to facilitate insertion of the device via pushing into the tissue pocket in the eye by the surgeon or practitioner, and thus, any device with a higher surface friction would experience resistance against the pushing motion which would likely lead to deformation of the device during implantation, which likely would collapse (e.g., flex, fold, buckle, deform, or crumple) the device.

In the aforementioned Ahmed® model, the plate body 652 is made of medical grade silicone, and the casing of the valve 656 is made of medical grade polypropylene.

The solid silicone plate body 652 does not integrate with the surrounding tissue when implanted, and it is believed that the lack of integration causes a poor tissue response to alleviate the stress caused by the pressure exerted by the plate body 652, leading to fibrosis that will plague the plate body 652, in turn causing high complication rates over subsequent years after the device is implanted as further explained in the article: Christakis P G et al. “The Ahmed Versus Baerveldt Study: Five-Year Treatment Outcomes.” Ophthalmology. 2016 October; 123(10):2093-102. DOI: 10.1016/j.ophtha.2016.06.035. Epub 2016 Aug. 17. PMID: 27544023, which is incorporated herein by reference in its entirety for all purposes. For example, FIG. 2 of the article illustrates the cumulative failure rate of the Ahmed® model and the Baerveldt® Glaucoma Implant device model 101-350.

Furthermore, the valve 656 has a duckbill configuration that uses two rigid flaps that are pressed together (although only one is visible in FIG. 10A). Even if the plate body 652 was not rigid, the prior art device could not be bent, curved, or shaped without affecting the functionality of the valve 656. In comparison to some prior-art glaucoma drainage devices, the medical devices 100, 200, 300, 350, 500, 550 as presently disclosed offers such benefits as providing a reversibly deformable, or flexible, device that includes an inner channel that is both resealable and permits fluid passage under certain fluid pressures, optionally without requiring a metal or rigid casing to provide a compressive force. The reversibly deformable or flexible nature of the medical devices 100, 200, 300, 350, 500, 550 (e.g., the tube 102 and the occluding member 104) provides better comfort for the patient than the prior-art devices when the device is at least partially implanted in the eye as explained above. Various embodiments of the medical device 100, 200, 300, 350, 500, 550 as presently disclosed may also have smaller thickness than the prior-art devices as shown in FIGS. 10A-10C. Furthermore, by providing an occluding member 104, 204, 304, 354, 504, 554 (which are effectively operable as a valve) inside the tube 102, 202, 302, 352, 502, 552, the medical devices 100, 200, 300, 350, 500, 550 eliminate the need to install the valve V at the end of the drainage tube T as shown in the aforementioned prior-art examples.

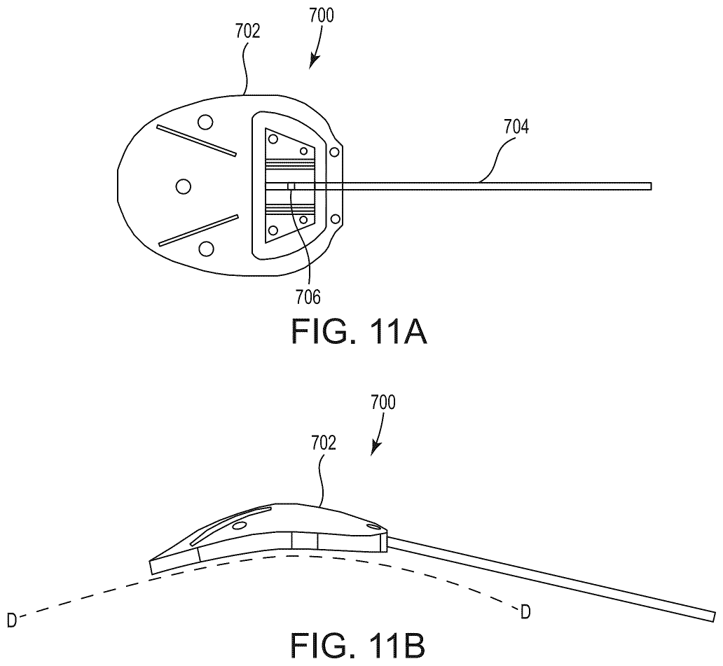

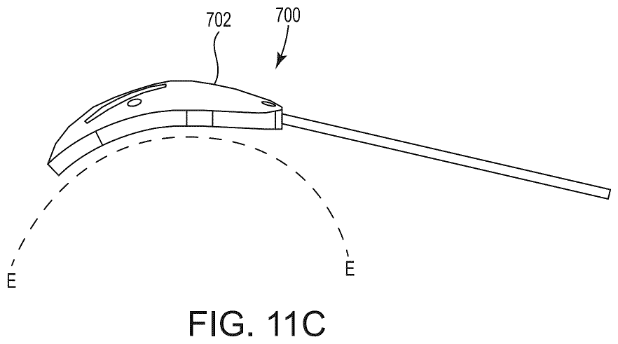

FIGS. 11A-11C illustrate a glaucoma drainage device 700 that is similar to the prior art example shown in FIGS. 10A-10C. However, the glaucoma drainage device includes a flexible plate body 702. The plate body 702 is allowed to be flexible because a tube 704 includes a valve 706 of the configuration of the present disclosure (e.g., the valve 706 is flexible because it has the configuration of, for example, one of the medical devices 100, 200, 300, 350, 500, or 550). As such, the plate body 702 can assume the curvature D-D shown in FIG. 11B and the curvature E-E shown in FIG. 11C.

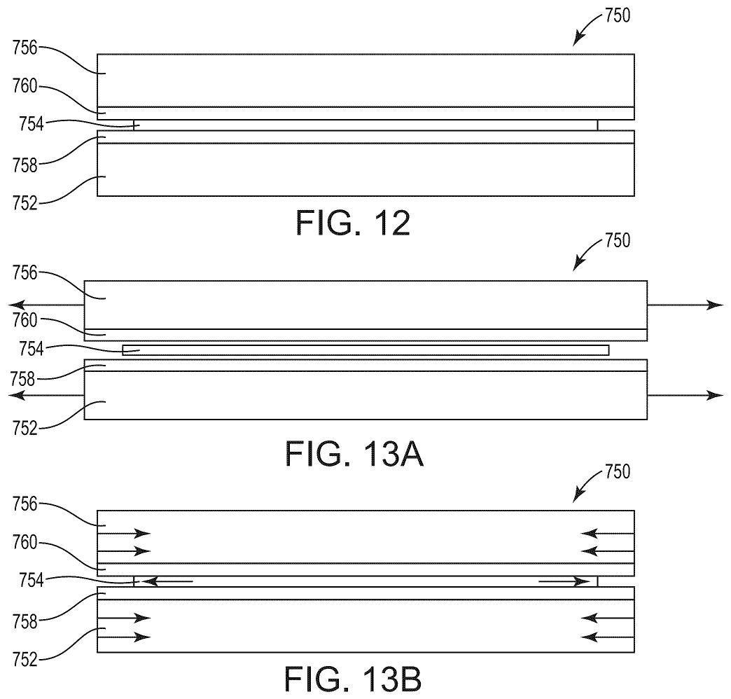

FIG. 12 is a schematic diagram of an alternative medical device 750. In some examples, the medical device 750 is a septum that comprises several layers that are stacked with adhesive in between. In some examples, the bottom layer 752 is a thick layer of elastomeric material (e.g., silicone), the seal layer 754 is a thin layer of elastomeric material (e.g., silicone that is the same as or different than that of the bottom layer 752), and the top layer 756 is a thick layer of elastomeric material (e.g., silicone that is the same as or different than that of the bottom layer 752 and/or the seal layer 754). In some examples, compared to the thicknesses (i.e., the vertical height as shown in FIG. 12) of the layers 752, 756, a thickness of the seal layer 754 is less than about 50%, less than about 40%, or less than about 20% as thick, The layers 752, 754 are adhered together using an adhesive layer 758, and the layers 754, 756 are adhered together using an adhesive layer 760 (which is the same as or different from the adhesive layer 758).

FIG. 13A is a schematic diagram of an operation of manufacturing the medical device 750. In some examples, the layers 752, 756 are laterally (i.e., horizontally, according to the orientation of FIG. 13A) tensioned and stretched (as indicated by the outward-facing arrows) before applying the adhesive layers 758, 760. In some examples, compared to their natural (i.e., unstrained) widths, the adhesive layers 758, 760 are stretched to be at least about 5%, at least about 10%, or at least about 25% wider. In some examples, the seal layer 754 is positioned in between the layers 752, 756 without being under tension. The five layers 752-760 are then longitudinally (i.e., vertically, according to the orientation of FIG. 13A) pressed together and the adhesive layers 758, 760 are cured.

The curing of the adhesive layers 758, 760 occurs while the layers 752, 756 are still in tension. Once the layers 752, 756 are released they will contract and compress the seal layer 754 until the medical device 750 reaches an equilibrium of the forces that are shown in FIG. 13B (as indicated by the arrows). More specifically, the seal layer 754 exerts an outward force on the layers 752, 756 which are both exerting an inward force on the seal layer 754. The result is that the layers 752, 756 will still be in some tension (albeit less than they were in prior to the curing of the adhesive layers 758, 760), and the seal layer 754 will be in compression (as opposed to the neutral state it was in prior to the curing of the adhesive layers 758, 760). Thus, the medical device 750 can function as a stopper and/or a pressure regulating septum.

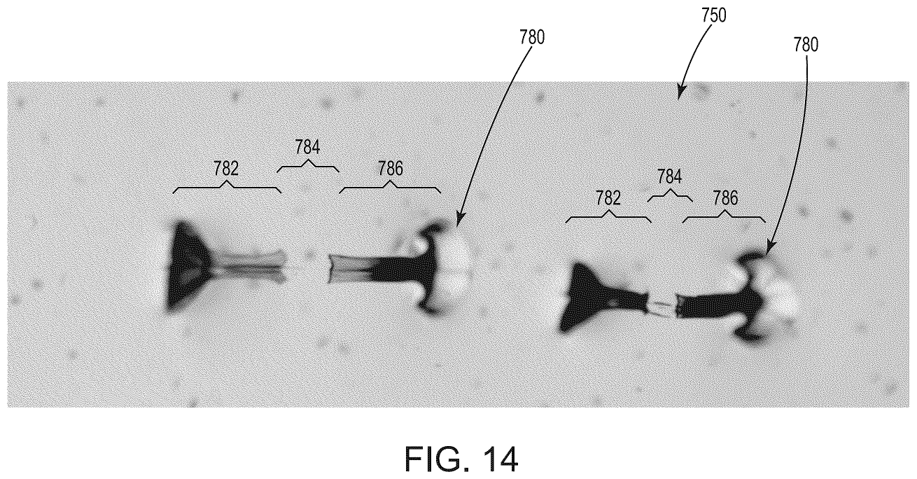

FIG. 14 is a photograph of a perspective view of the medical device 750 after having been punctured in two locations, forming two channels 780. Each channel 780 has three regions 782, 784, 786 that are located along its longitudinal length (i.e. left-to-right and into-and-out-of the page, according to the orientation of FIG. 14). The regions 782 are located in the bottom layer 752 (shown in FIG. 13B), the regions 784 are located in the seal layer 754 (shown in FIG. 13B), and the regions 786 are located in the top layer 756 (shown in FIG. 13B).

In some examples, the seal layer 754 is in compression, so the regions 784 of the channels 780 are closed and are nearly invisible to the naked eye. In contrast, the layers 752, 756 are in tension, so the regions 782, 786 of the channels 780 are enlarged. While the enlargement allows fluid to enter into the regions 782, 786 more easily, the regions 784 will determine the water leak pressure of the medical device 750 since the channels 780 are the most compressed therein.

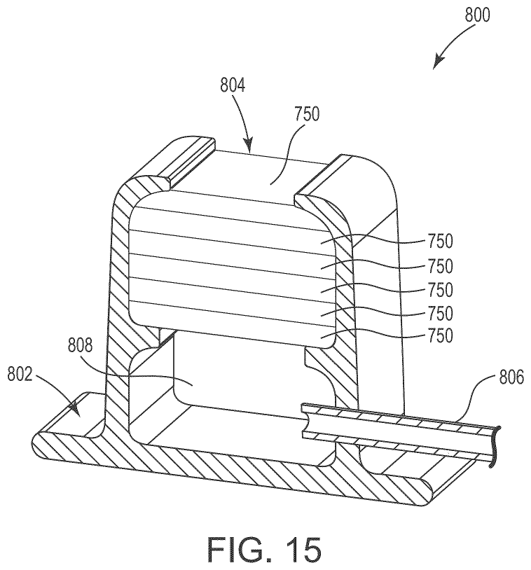

FIG. 15 is a schematic cross-sectional slice of an alternative port-a-cath device 800. In some examples, the port-a-cath device 800 includes a solid or rigid casing 802, an access point 804 positioned in the casing 802, a catheter 806 extending through the casing 802, and an internal chamber 808 that is defined by the casing 802, the access point 804, and the catheter 806. In some examples, the access point 804 is comprised of a plurality of stacked medical devices 750, although in other examples, there is only a single medical device 750. In some of the examples where there are multiple medical devices 750, the medical devices 750 are manufactured individually and then each is positioned in the casing 802. In some of the examples where there are multiple medical devices 750, the medical devices 750 are manufactured individually, then adhered together in a stack, and then the stack is positioned in the casing 802. In some of the examples where there is a single medical device 750, the medical device 750 can have more layers than just the five discussed with respect to FIG. 12. In some such examples, there are multiple seal layers 754 positioned between the bottom layer 752 and the top layer 756, with an intermediate layer (not shown) between each seal layer 754. In some examples, the intermediate layers are similar to or the same as the bottom layer 752 and/or the top layer 756.

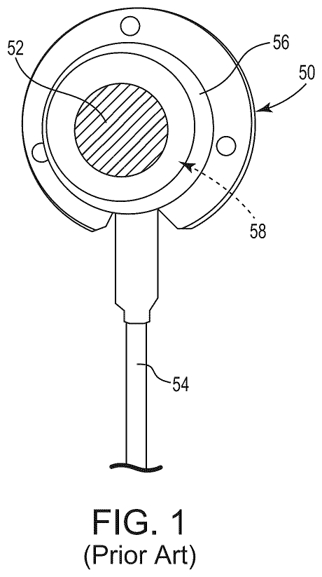

The access point 804 acts as a septum for the port-a-cath device 800, and the access point 804 is configured to be puncturable by, for example, a needle (not shown). In contrast with the port-a-cath 50 (shown in FIG. 1), the casing 802 of the port-a-cath 800 does not need to exert much, if any, pressure to compress the access point 804 to inhibit leakage from the internal chamber 808 because the resealing of the access point 804 is performed by the internal stresses in each of the medical devices 750.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A medical device for controlling a flow of a fluid passing through the device, the medical device comprising:
a tube having a tube length between opposing first and second tube ends and defining a tube axis of the tube, the first tube end having a first port and the second tube end having a second port, the first and second ports each communicating with a tube lumen extending within the tube between the first and second ports, the tube lumen defining a lumen diameter of the tube, the tube being formed from a first elastomeric material sufficiently rigid to maintain at least a portion of the lumen diameter and sufficiently flexible to define an adjustable curvature of the tube lumen disposed along the tube axis; and
an occluding member fixedly disposed within the tube lumen between the first and second ports, the occluding member being formed from a second elastomeric material maintained in a compressed state by a compressive force from the tube, the second elastomeric material applying an expansive force against the tube within the tube lumen along at least a portion of the tube length,
wherein the occluding member further includes an internal channel extending through the occluding member between the first and second ports, the internal channel having a closed state maintained by the compressive force to collapse the internal channel to prevent the flow of the fluid through the internal channel and an open state overcoming the compressive force to at least partially open the internal channel to permit the flow of the fluid through the internal channel, and
wherein in the closed state the fluid disposed at the first port is at a first pressure that does not overcome the compressive force applied to the internal channel, and wherein in the open state the fluid disposed at the first port is at a greater second pressure that overcomes the compressive force applied to the internal channel.

2. The medical device of claim 1, wherein the first and second elastomeric materials are different forms of a same material.

3. The medical device of claim 1, wherein the first elastomeric material defines an internal surface adjacent to the second elastomeric material.

4. The medical device of claim 1, wherein when the tube is cut to form a cut surface, the second elastomeric material at the cut surface protrudes axially outwardly at a greater degree than the first elastomeric material at the cut surface.

5. A flow-controlling medical device for controlling a flow of a fluid through the device, the device comprising:
a containment member defining a length extending between opposing ends of the containment member, the containment member further defining an inner lumen of the containment member extending between the opposing ends; and
an occluding member disposed to occlude the inner lumen of the containment member, the occluding member being resealable such that a channel formed by a puncturing force applied to the occluding member will reseal to inhibit a passage of the fluid through the channel once the puncturing force is removed,
wherein the containment member is deformable to form a bent configuration of the device that maintains a resealability of the occluding member.

6. The medical device of claim 5, wherein the containment member is deformable in that the medical device is reversibly deformable and is capable of returning to an initial configuration of the medical device.

7. The medical device of claim 5, wherein the bent configuration is a deformed configuration that maintains flow-controlling properties of the medical device.

8. The medical device of claim 5, wherein the bent configuration is nondestructive to an initial configuration of the medical device prior to the containment member being deformed to form the bent configuration of the device.

9. The medical device of claim 5, wherein the occluding member is configured to be pressurized by the containment member.

10. The medical device of claim 5, wherein the containment member and the occluding member are dyed using different colors to visually distinguish between the containment member and the occluding member.

11. The medical device of claim 5, wherein an end portion of the medical device includes a guide member to visually distinguish between the occluding member and the containment member.

12. A flow-controlling medical device for controlling a flow of a fluid through the device, the device comprising:
a containment member defining a length extending between opposing ends of the containment member, the containment member further defining an inner lumen of the containment member extending between the opposing ends; and
an occluding member disposed to occlude the inner lumen of the containment member, the occluding member being resealable such that a channel formed by a puncturing force applied to the occluding member will reseal to inhibit a passage of the fluid through the channel when the puncturing force is removed,
wherein the occluding member includes a preformed internal channel extending through the occluding member and having a closed state where the internal channel is collapsed to prevent the flow of the fluid through the internal channel and an open state where the internal channel is opened to permit the flow of the fluid through the internal channel, and
wherein the closed state is maintained when the fluid disposed at one of the opposing ends of the containment member is at a first pressure insufficient to open the internal channel, and wherein the open state is maintained when the fluid disposed at the one of the opposing ends of the containment member is at a second pressure sufficient to open the internal channel.

13. The medical device of claim 12, wherein the second pressure is a calibrated pressure that opens the internal channel upon reaching a threshold pressure.

14. The medical device of claim 12, wherein the occluding member is configured to be pressurized by the containment member.

15. The medical device of claim 12, wherein the containment member is deformable to form a bent configuration of the device that maintains a resealability of the occluding member.

16. The medical device of claim 15, wherein the containment member is deformable in that the medical device is reversibly deformable and is capable of returning to an initial configuration of the medical device.

17. The medical device of claim 15, wherein the bent configuration is a deformed configuration that maintains flow-controlling properties of the medical device.

18. The medical device of claim 15, wherein the bent configuration is nondestructive to an initial configuration of the medical device prior to the containment member being deformed to form the bent configuration of the device.

19. The medical device of claim 12, wherein the containment member and the occluding member are dyed using different colors to visually distinguish between the containment member and the occluding member.

20. The medical device of claim 12, wherein an end portion of the medical device includes a guide member to visually distinguish between the containment member and the occluding member.
